(19) United States
(12) Patent Application Publication
(10) Pub. No.: US 2018/0036319 A1
(43) Pub. Date: Feb. 8, 2018

(54) TRANSDERMAL DRUG DELIVERY SYSTEMS FOR NORETHINDRONE ACETATE

(71) Applicant: Noven Pharmaceuticals, Inc., Miami, FL (US)

(72) Inventor: Viet NGUYEN, Miami, FL (US)

(73) Assignee: Noven Pharmaceuticals, Inc., Miami, FL (US)

(21) Appl. No.: 15/667,216

(22) Filed: Aug. 2, 2017

Related U.S. Application Data
(60) Provisional application No. 62/370,346, filed on Aug. 3, 2016.

(51) Int. Cl. A61K 31/569; A61K 9/70; A61K 47/10

(52) U.S. Cl. CPC A61K 31/569; A61K 47/10; A61K 9/7069; A61K 9/7053

(57) ABSTRACT

Described are transdermal drug delivery systems for the transdermal administration of norethindrone acetate in the form of a flexible finite system for topical application, comprising a drug-in-adhesive polymer matrix comprising norethindrone acetate (NETA) as the only systemically active drug, an adhesive polymer selected from silicone adhesives and polyisobutylene (PIB) adhesives, a penetration enhancer, and a crystallization inhibitor. Methods of making and using the systems also are described.

### CROSS-REFERENCE TO RELATED PATENT APPLICATIONS

This application claims the priority benefits under 35 USC §119(e) to U.S. provisional application 62/370,346, filed Aug. 3, 2016, the entire contents of which are incorporated herein by reference in their entirety.

### FIELD OF THE INVENTION

Described herein are compositions and methods for the transdermal delivery of norethindrone acetate (NETA). The compositions and methods are useful, for example, for contraception.

### BACKGROUND

Transdermal delivery systems (e.g., patches) as dosage forms have been the subject of a vast number of patent applications over the last 25 years, yielding many patents but few commercial products in comparison. To those working in the field, the relatively small number of commercial products is not surprising. Although regulatory, economic, and market hurdles play a role in limiting the number of products on the market, the task of developing a transdermal delivery system that achieves desired physical and pharmacokinetic parameters to satisfy physician and patient demand is more daunting. Parameters to be considered during commercial product development may include drug solubility, drug stability (e.g., as may arise from interaction with other component materials and/or the environment), delivery of a therapeutic amount of drug at a desired delivery rate over the intended duration of use, adequate adhesion at the anatomical site of application, integrity (e.g., minimal curling, wrinkling, delaminating and slippage) with minimal discomfort, irritation and sensitization both during use and during and after removal, and minimal residual adhesive (or other components) after removal. Size also may be important from a manufacturing and patient viewpoint, and appearance may be important from a patient viewpoint.

A transdermal drug delivery system comprising both norethindrone acetate and estradiol (CombiPatch®, Noven Pharmaceuticals, Inc.) is known, but currently there is no U.S. FDA-approved transdermal drug delivery system for only norethindrone acetate. Thus, there remains a need for transdermal drug delivery systems designed for the delivery of norethindrone acetate.

### SUMMARY

In accordance with some embodiments, there are provided transdermal compositions for the transdermal delivery of norethindrone acetate in the form of a flexible finite system for topical application, comprising a drug-in-adhesive polymer matrix comprising norethindrone acetate as the only systemically active drug, an adhesive polymer selected from silicone adhesives and polyisobutylene (PIB) adhesives, a penetration enhancer, and a crystallization inhibitor.

In accordance with any of the embodiments described herein, the penetration enhancer may comprise any one or more of oleyl alcohol, dipropylene glycol, isopropyl myristate, and glyceryl monooleate, such as oleyl alcohol; oleyl alcohol and dipropylene glycol; oleyl alcohol and glyceryl monooleate; or dipropylene glycol, isopropyl myristate, and glyceryl monooleate.

In accordance with any of the embodiments described herein, the crystallization inhibitor may comprise one or more of povidone, polyvinylpyrrolidone (PVP), copovidone, polyvinylpyrrolidone-vinyl acetate copolymer (PVP/VA), crospovidone, or polyvinylpolypyrrolidone (PVPP).

In accordance with any of the embodiments described herein, the polymer matrix may comprise an amount of norethindrone acetate sufficient to deliver a pharmaceutically effect amount of norethindrone acetate for contraception, such as at least about 0.14 mg/day norethindrone acetate, or at least about 0.25 mg/day norethindrone acetate.

In accordance with any of the embodiments described herein, the polymer matrix may comprise an amount of norethindrone acetate sufficient to achieve sustained delivery of norethindrone acetate over a period of time of at least 3 days, at least 4 days, or at least 7 days. In some embodiments, the amount of norethindrone acetate is sufficient to deliver a pharmaceutically effective amount of norethindrone acetate for contraception, or at least about 0.14 mg/day or at least about 0.25 mg/day norethindrone acetate, over a period of time of at least 3 days, at least 4 days, or at least 7 days.

In accordance with any of the embodiments described herein, the polymer matrix may further comprise one or more acrylic pressure-sensitive adhesive polymers, such as one or more non-functional acrylic polymers, hydroxyl-functional acrylic polymers and/or amide-functional acrylic polymers.

In accordance with some embodiments, the systems are monolithic systems. In accordance with other embodiments, the systems comprise a face adhesive layer, such as a polyisobutylene face adhesive layer.

In accordance with any of the embodiments described herein, the transdermal drug delivery system may further comprise a backing layer and/or a release liner.

In accordance with some embodiments, there are provided methods of preparing a transdermal composition for the transdermal delivery of norethindrone acetate in the form of a flexible finite system for topical application, comprising preparing a drug-in-adhesive polymer matrix comprising norethindrone acetate as the only systemically active drug, a silicone adhesive or a polyisobutylene (PIB) adhesive, a penetration enhancer, and a crystallization inhibitor.

In accordance with some embodiments, there are provided methods of transdermally delivering norethindrone acetate comprising applying a transdermal drug delivery system as described herein to the skin or mucosa of a subject in need thereof. In some embodiments, the subject is a human female subject. In some embodiments, the method is for contraception. In some embodiments, the transdermal drug delivery system is applied for a duration of at least 3 days, at least 4 days, or at least 7 days.

In accordance with some embodiments, there are provided transdermal drug delivery systems as described herein for providing contraception. In some embodiments, the system is formulated to deliver a pharmaceutically effective amount of norethindrone acetate for contraception, or at least about 0.14 mg/day, or at least about 0.25 mg/day norethindrone acetate over a period of at least 3 days, at least 4 days, or at least 7 days.

In accordance with some embodiments, there are provided uses of norethindrone acetate in the preparation of transdermal drug delivery systems as described herein for providing contraception. In some embodiments, the system is formulated to deliver a pharmaceutically effective amount of norethindrone acetate for contraception, or at least about 0.14 mg/day, or at least about 0.25 mg/day norethindrone acetate over a period of at least 3 days, at least 4 days, or at least 7 days.

Also provided are methods of providing contraception to a female subject in need thereof, comprising applying a transdermal drug delivery system as described herein to the skin or mucosa of a subject in need thereof, wherein the system is formulated to deliver a pharmaceutically effective amount of norethindrone acetate for contraception, or at least about 0.14 mg/day, or at least about 0.25 mg/day norethindrone acetate over a period of at least 3 days, at least 4 days, or at least 7 days, and is applied for a period of at least 3 days, at least 4 days, or at least 7 days, respectively.

### BRIEF DESCRIPTION OF THE DRAWINGS

FIG. 1A illustrates a monolithic transdermal delivery system as described herein that consists of a backing, a drug-in-adhesive matrix layer, and a release liner (when present). FIG. 1B illustrates a transdermal delivery system as described herein that further comprises a face adhesive layer between the drug-in-adhesive matrix layer and release liner (when present).

FIG. 2 shows the results of a 7-day in vitro flux study from monolithic systems having polymer matrices based on a silicone adhesive as described herein, with or without an acrylic adhesive.

FIG. 3 shows the results of a 7-day in vitro flux study from a monolithic system having a polymer matrix based on a PIB adhesive as described herein, as compared to CombiPatch®.

FIG. 4 shows the results of a 7-day in vitro flux study from systems having a polymer matrix based on a PIB adhesive as described herein, without or with a face adhesive layer, as compared to CombiPatch®.

FIG. 5 shows the results of a 7-day in vitro flux study from a system having a polymer matrix based on a PIB adhesive as described herein, with a face adhesive layer, as compared to CombiPatch®.

FIG. 6 shows the results of a 7-day in vitro flux study from a system having a polymer matrix as described herein, based on a PIB adhesive and also having an acrylic-based polymer, as compared to CombiPatch®.

FIG. 7 shows the results of a 7-day in vitro flux study from a system having a polymer matrix as described herein, based on a PIB adhesive and also having an acrylic-based polymer, as compared to CombiPatch®.

### DETAILED DESCRIPTION

Described herein are compositions and methods for the transdermal delivery of norethindrone acetate (NETA). The compositions and methods are useful, for example, for contraception. In some embodiments the compositions are in the form of a flexible finite system for topical application, comprising a drug-in-adhesive polymer matrix comprising norethindrone acetate, an adhesive polymer, a penetration enhancer, and a crystallization inhibitor.

The drug-containing polymer matrix of CombiPatch® includes acrylic adhesive, silicone adhesive, oleic acid, dipropylene glycol, and povidone. Since the presence of another drug (e.g., estradiol) in the transdermal composition may impact physical and pharmacodynamics properties, a new polymer matrix had to be developed for transdermal delivery of norethindrone acetate without estradiol.

The present inventors surprisingly discovered that the compositions described herein are able to achieve the difficult goal of providing sustained delivery of norethindrone acetate at therapeutically effective levels. Typically, achieving sustained delivery and achieving a sustained delivery rate are competing goals, with delivery rate decreasing as the delivery period increases. Therefore, it was a challenge to arrive at compositions that can achieve sustained delivery at a sustained delivery rate.

### Definitions

Technical and scientific terms used herein have the meanings commonly understood by one of ordinary skill in the art to which the present invention pertains, unless otherwise defined. Reference is made herein to various methodologies known to those of ordinary skill in the art.

Publications and other materials setting forth such known methodologies to which reference is made are incorporated herein by reference in their entireties as though set forth in full. Any suitable materials and/or methods known to those of ordinary skill in the art can be utilized in carrying out the present invention. However, specific materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” means that the number comprehended is not limited to the exact number set forth, and refers to ranges substantially around the stated value while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The phrase “substantially free” as used herein generally means that the described composition (e.g., transdermal drug delivery system, polymer matrix, etc.) comprises less than about 5%, less than about 3%, or less than about 1% by weight, based on the total weight of the composition at issue, of the excluded component.

The phrase “free of” as used herein means that the described composition (e.g., polymer matrix, etc.) is formulated without adding the excluded component(s) as an intended component, although trace amounts may be present in other components or as a by-product or contaminant, such that the composition comprises at most only trace amounts of the excluded component(s). In some embodiments, the compositions and systems described herein are formulated without any other systemically active drug other than norethindrone acetate.

As used herein “subject” denotes any animal in need of drug therapy, including humans. For example, a subject may be suffering from or at risk of developing a condition that can be treated or prevented with norethindrone acetate, or may be taking norethindrone acetate for health maintenance purposes. In specific embodiments, the subject is a female subject taking norethindrone acetate for contraceptive purposes.

As used herein, the phrases “therapeutically effective amount” and “therapeutic level” mean that drug dosage or plasma concentration in a subject, respectively, that provides the specific pharmacological response for which the drug is administered in a subject in need of such treatment, such as contraception. It is emphasized that a therapeutically effective amount or therapeutic level of a drug will not always be effective in treating the conditions/diseases described herein, even though such dosage is deemed to be a therapeutically effective amount by those of skill in the art. For convenience only, exemplary dosages, drug delivery amounts, therapeutically effective amounts and therapeutic levels are provided below with reference to adult human subjects. Those skilled in the art can adjust such amounts in accordance with standard practices as needed to treat a specific subject and/or condition/disease. In some embodiments, a therapeutically effect amount of norethindrone acetate for contraception is about 0.14 mg/day or 0.25 mg/day.

As used herein, “active surface area” means the surface area of the drug-containing layer of the transdermal drug delivery system.

As used herein, “coat weight” refers to the weight of the drug-containing layer per unit area of the active surface area of the transdermal drug delivery system.

As used herein, “flux” (also called “permeation rate”) is defined as the absorption of a drug through skin or mucosal tissue, and is described by Fick's first law of diffusion:

J=−D(dCm/dx)

where J is the flux in g/cm²/hr, D is the diffusion coefficient of the drug through the skin or mucosa in cm²/hr and dCm/dx is the concentration gradient of the drug across the skin or mucosa.

As used herein, the term “transdermal” refers to delivery, administration or application of a drug by means of direct contact with skin or mucosa. Such delivery, administration or application is also known as dermal, percutaneous, transmucosal and buccal. As used herein, “dermal” includes skin and mucosa, which includes oral, buccal, nasal, rectal and vaginal mucosa.

As used herein, “transdermal drug delivery system” refers to a system (e.g., a device) comprising a composition that releases drug upon application to the skin (or any other surface noted above). A transdermal drug delivery system may comprise a drug-containing layer, and, optionally, a backing layer and/or a release liner layer. In some embodiments, the transdermal drug delivery system is a substantially non-aqueous, solid form, capable of conforming to and adhering to the surface with which it comes into contact, and capable of maintaining such contact so as to facilitate topical application without adverse physiological response, and without being appreciably decomposed by aqueous contact during topical application to a subject. Many such systems are known in the art and commercially available, such as transdermal drug delivery patches. As described below, in some embodiments, a transdermal drug delivery system comprises a drug-containing polymer matrix that comprises a pressure-sensitive adhesive or bioadhesive, and is adopted for direct application to a user's (e.g., a subject's) skin. In other embodiments, the polymer matrix is non-adhesive and may be provided with separate adhesion means (such as a separate adhesive layer) for application and adherence to the user's skin. In some embodiments, the transdermal drug delivery system comprises a skin-contacting face adhesive layer. Additionally or alternatively, the system may comprise a rate-controlling membrane.

As used herein, “polymer matrix” refers to a polymer composition which contains one or more drugs. In some embodiments, the matrix comprises a pressure-sensitive adhesive polymer or a bioadhesive polymer. In other embodiments, the matrix does not comprise a pressure-sensitive adhesive or bioadhesive. As used herein, a polymer is an “adhesive” if it has the properties of an adhesive per se, or if it functions as an adhesive by the addition of tackifiers, plasticizers, crosslinking agents or other additives. Thus, in some embodiments, the polymer matrix comprises a pressure-sensitive adhesive polymer or a bioadhesive polymer, with drug dissolved or dispersed therein. The polymer matrix also may comprise tackifiers, plasticizers, crosslinking agents, enhancers, co-solvents, fillers, antioxidants, solubilizers, crystallization inhibitors, or other additives described herein.

As used herein, the term “pressure-sensitive adhesive” refers to a viscoelastic material which adheres instantaneously to most substrates with the application of very slight pressure and remains permanently tacky. A polymer is a pressure-sensitive adhesive within the meaning of the term as used herein if it has the properties of a pressure-sensitive adhesive per se or functions as a pressure-sensitive adhesive by admixture with tackifiers, plasticizers or other additives.

The term pressure-sensitive adhesive also includes mixtures of different polymers and mixtures of polymers, such as polyisobutylenes (PIB), of different molecular weights, wherein each resultant mixture is a pressure-sensitive adhesive. In the last case, the polymers of lower molecular weight in the mixture are not considered to be “tackifiers,” said term being reserved for additives which differ other than in molecular weight from the polymers to which they are added.

In some embodiments, the polymer matrix is a pressure-sensitive adhesive at room temperature and has other desirable characteristics for adhesives used in the transdermal drug delivery art. Such characteristics include good adherence to skin, ability to be peeled or otherwise removed without substantial trauma to the skin, retention of tack with aging, etc. In some embodiments, the polymer matrix has a glass transition temperature (Tg), measured using a differential scanning calorimeter, of between about −70° C. and 0° C.

As used herein, the term “rubber-based pressure-sensitive adhesive” refers to a viscoelastic material which has the properties of a pressure-sensitive adhesive and which contains at least one natural or synthetic elastomeric polymer.

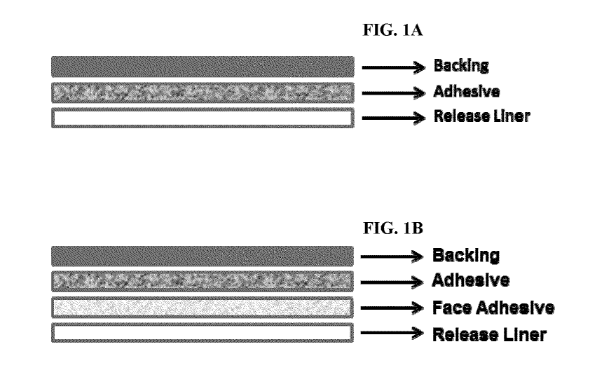

In some embodiments, the transdermal drug delivery system includes one or more additional layers, such as one or more additional polymer matrix layers, or one or more adhesive layers that adhere the transdermal drug delivery system to the user's skin, such as a face adhesive layer, as illustrated in FIG. 1B. In other embodiments, the transdermal drug delivery system is monolithic, meaning that it comprises a single polymer matrix layer comprising a pressure-sensitive adhesive or bioadhesive with drug dissolved or dispersed therein, and no face adhesive, no rate-controlling membrane, no other polymeric adhesive layer and no other drug-containing layer.

A transdermal drug delivery system may include a drug impermeable backing layer or film. (By “impermeable” to the drug is meant that no substantial amount of drug loss through the backing layer is observed) The backing layer protects the polymer matrix from the environment and prevents loss of the drug and/or release of other components to the environment during use. Materials suitable for use as backing layers are well-known in the art and commercially available, such as films of polyester, polyethylene, vinyl acetate resins, ethylene/vinyl acetate copolymers, polyvinyl chloride, polyurethane, and the like, metal foils, non-woven fabric, cloth and commercially available laminates. A typical backing material has a thickness in the range of 2 to 1000 micrometers. For example, 3M's Scotch Pak™ 1012 or 9732 (a polyester film with an ethylene vinyl acetate copolymer heat seal layer), 9723 (a laminate of polyethylene and polyester), or CoTran 9720 (a polyethylene film) are useful in the transdermal drug delivery systems described herein, as are Dow® backing layer films, such as Dow® BLF 2550 (a multi-layer backing comprising ethylene vinyl acetate layers and an internal vinylidene chloride/methyl acrylate layer).

The transdermal drug delivery system also may include a release liner, typically located adjacent the opposite face of the system as compared to the backing layer. When present, the release liner is removed from the system prior to use to expose the polymer matrix layer and/or an adhesive layer prior to topical application. Materials suitable for use as release liners are well-known known in the art and include silicone- or fluorocarbon-coated polyester release liners, such as the commercially available products of Dow Corning Corporation designated Bio-Release® liner and Syl-off® 7610, Loparex's PET release liner (silicone-coated) and 3M's 1020, 1022, 9741, 9744, 9748, 9749 and 9755 Scotchpak™ (fluoropolymer-coated polyester films).

A transdermal drug delivery system may be packaged or provided in a package, such as a pouchstock material used for transdermal drug delivery systems in general. For example, DuPont's Surlyn® can be used in a pouchstock material. Alternatively, a pouchstock comprising a coextruded ethylene acrylic acid/low-density polyethylene (EAA/LDPE) material, or Barex® from INEOS (acrylonitrile—methyl acrylate) may be used.

As used herein, a “monolithic” transdermal drug delivery system may include a backing layer and/or release liner, as illustrated in FIG. 1A, and may be provided in a package.

Norehtindrone Acetate

Norethindrone acetate (NETA) is a white solid, chemically described as 17-hydroxy-19-nor-17α-pregn-4-en-20-yn-3-one acetate. The molecular weight of NETA is 340.47 and the molecular formula is C₂₂H₂₈O₃. The structural formula NETA is:

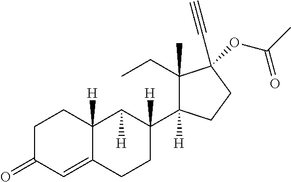

As noted above, in some embodiments the systems and compositions described herein include NETA as the only systemically active drug. In accordance with such embodiments, the systems and compositions are free of other hormonal agents, including other progestagens and estrogens, including estradiol. That is, in embodiments where the systems and compositions include NETA as the only systemically active drug, such systems and compositions are free of estradiol, free of estrogens, and free of other progestagens.

The amount of NETA to be incorporated in the polymer matrix of the systems described herein varies depending on the desired therapeutic effect, and the time span for which the system is to provide therapy. For most drugs, the passage of the drugs through the skin will be the rate-limiting step in delivery. A minimum amount of drug in the system is selected based on the amount of drug which passes through the skin in the time span for which the system is to provide therapy. In some embodiments, a system for the transdermal delivery of NETA is used over a period of at least about 1 day, 3 days, 7 days, or longer. Thus, in some embodiments, the systems comprise an amount of NETA sufficient to deliver a therapeutically effective amount over a period of from 1 day to 3 days, 3 days to 7 days, or longer, including for 1 day, for 2 days, for 3 days, for 4 days, for 5 days, for 6 days, for 7 days, or for longer.

The CombiPatch® product delivers about 0.14 mg/day NETA from a 9 cm² round patch, and about 0.24 mg/day NETA from a 16 cm² round patch. In some embodiments, the systems described herein are formulated to provide at least about 0.015 mg/cm²/day (at least about 15.0 μg/cm²/day, including at least about 0.0155 mg/cm²/day (at least about 15.5 μg/cm²/day).

In some embodiments, the polymer matrix comprises from about 1% to about 10% by weight NETA, including from about 1% to about 5%, including about 1%, about 1.5%, about 2%, about 2.5%, about 3%, about 3.5%, about 4%, about 4.5%, or about 5% by weight NETA, based on the dry weight of the polymer matrix. In specific embodiments, a system for the transdermal delivery of NETA includes an amount of NETA effective to deliver a pharmaceutically effective amount of norethindrone acetate for contraception, or at least about 0.14 mg/day, or at least about 0.25 mg/day NETA.

Polymer Matrix

As noted above, in some embodiments the compositions described herein are in the form of a flexible finite system for topical application, comprising a drug-in-adhesive polymer matrix comprising NETA, an adhesive polymer selected from silicone adhesive polymers and polyisobutylene (PIB) adhesive polymers, a penetration enhancer, and a crystallization inhibitor and, optionally, a backing and a release liner.

Silicone Polymers

Suitable silicone polymers include silicone pressure-sensitive adhesives and silicone-based polymers that function as an adhesive by the addition of tackifiers, plasticizers, crosslinking agents, or other additives, including those known for use in transdermal drug delivery systems. The term “silicone polymer” is used herein interchangeably with the terms silicon polymer, siloxane, polysiloxane, and silicones, as is known in the art. Suitable polysiloxanes include silicone pressure-sensitive adhesives which are based on two major components: (i) a polymer or gum and (ii) a tackifying resin. A polysiloxane adhesive can be prepared by cross-linking a gum, typically a high molecular weight polydiorganosiloxane, with a resin, to produce a three-dimensional silicate structure, via a condensation reaction in an appropriate organic, volatile solvent, such as ethyl acetate or heptane. The ratio of resin to polymer can be adjusted in order to modify the physical properties of polysiloxane adhesives. See, e.g., Sobieski, et al., “Silicone Pressure Sensitive Adhesives,” Handbook of Pressure-Sensitive Adhesive Technology, 2nd ed., pp. 508-517 (D. Satas, ed.), Van Nostrand Reinhold, N.Y. (1989). Illustrative examples of silicone polymers having reduced silanol concentrations include silicone adhesives (and capped polysiloxane adhesives) such as those described in U.S. Pat. No. Re. 35,474 and U.S. Pat. No. 6,337,086, which are commercially available such as BIO-PSA® 7-4100, -4200 and -4300 product series (Dow Corning Corporation, Michigan), and non-sensitizing, pressure-sensitive adhesives produced with compatible organic volatile solvents (such as ethyl acetate or heptane) and available commercially such as BIO-PSA® 7-4400 series, -4500 series, such as BIO-PSA®-4502, and -4600 series. Further details and examples of silicone pressure-sensitive adhesives that can be used are mentioned in the following U.S. Pat. Nos. 4,591,622; 4,584,355; 4,585,836; and 4,655,767. Silicone fluids also are contemplated for use in the polymer matrices and methods described herein.

In embodiments comprising a silicone polymer, one or more silicone polymers can be used, and the silicone polymer(s) may comprise from about 50% to about 90% by weight of the polymer matrix, including from about 60% to about 75%, including about 60%, about 65% about 70% or about 75% by weight, based on the dry weight of the polymer matrix, e.g., not including the weight of processing solvents that are removed during the manufacturing process. In some embodiments, the amount of silicone polymer is selected to increase drug flux, reduce required drug loading and/or maximize efficiency of the composition.

In some embodiments comprising a silicone polymer, the composition may include more, less or about the same relative amount of silicone polymer than the polymer matrix of the CombiPatch® product. Additionally or alternatively, some embodiments comprising a silicone polymer may include more, less or about the same relative amount of hydroxy-functional acrylic polymer than the polymer matrix of the CombiPatch® product. Additionally or alternatively, some embodiments comprising a silicone polymer may include more, less or about the same relative amount of enhancer than the polymer matrix of the CombiPatch® product. Additionally or alternatively, some embodiments comprising a silicone polymer may include a different enhancer system than the polymer matrix of the CombiPatch® product. Thus, embodiments comprising a silicone polymer may include any one or more of more, less or about the same relative amount of silicone polymer; more, less or about the same relative amount of hydroxy-functional acrylic polymer; more, less or about the same relative amount of enhancer and/or a different enhancer system than the polymer matrix of the CombiPatch® product. In specific embodiments comprising a silicone polymer, the polymer matrix includes more relative amount of hydroxy-functional acrylic polymer than the polymer matrix of the CombiPatch® product and additionally or alternatively includes a different enhancer system, as illustrated in the examples below. In such embodiments, the relative amount of silicone polymer may or may not vary from that of the polymer matrix of the CombiPatch® product.

Polyisobutylene Polymers

Suitable PIB polymers include PIB pressure-sensitive adhesives known for use in transdermal drug delivery systems, including those sold by BASF under the Oppanol® B brand, which is a series of medium and high molecular weight polyisobutylene polymers having a weight-average molecular weight (MW) between 40,000 and 4,000,000, and include Oppanol® B100 and Oppanol® B11SFN, and those sold by Henkel Corporation, Bridgewater, N.J., such as DURO-TAK® 87-6906. In some embodiments, the polymer matrix comprises two or more polyisobutylene polymers of different molecular weights. In accordance with these embodiments, the relative amounts of polyisobutylene polymers can be selected and tailored to produce a product with satisfactory physical and pharmacokinetic properties.

In some embodiments, a PIB polymer is used with a tackifier. In other embodiments a PIB adhesive is used without a tackifier, such that the formulation is free of tackifiers, e.g., is formulated without a separate tackifier component. In this regard, those skilled in the art will understand that other components of the polymer matrix, such as the penetration enhancer, may exhibit tackifier properties. In some embodiments, the polymer matrix may include an acrylic polymer that acts as a tackifier, such as amide-group containing acrylic polymer, such as one or more of those discussed below (e.g., DURO-TAK® 87-900A).

Suitable tackifiers for use with PIB polymers in transdermal drug delivery systems are known in the art and include hydrocarbon resins, mineral oil, and hydrogenated polyisobutenes, such as Panalane® sold by Lipo Chemicals, Inc. (Paterson, N.J.). In some embodiments, the tackifier is a hydrogenated polyisobutenes, such as Panalane®. In some embodiments, the tackifier is a hydrogenated hydrocarbon resin.

In some embodiments comprising a PIB polymer, the matrix optionally may further include one or more modifiers such a silicone fluid (e.g., cyclomethicone) and SiO₂ or TiO₂, such as may be useful to improve cohesions (shear value) and/or decrease cold flow.

In embodiments comprising a PIB polymer, one or more PIB polymers can be used, and the PIB polymer(s) may comprise from about 50% to about 90% by weight of the polymer matrix, including from about 60% to about 75%, including about 60%, about 65% about 70% or about 75% by weight, based on the dry weight of the polymer matrix, e.g., not including the weight of processing solvents that are removed during the manufacturing process. In some embodiments, the amount of PIB polymer is selected to increase drug flux, reduce required drug loading and/or maximize efficiency of the composition.

Acrylic Polymers

In some embodiments, the polymer matrix includes an acrylic polymer, in addition to the silicone or PIB polymer discussed above. As illustrated in the examples, the use of an acrylic polymer can impact the drug delivery profile of NETA from the compositions described here. Thus, in some embodiments, the acrylic polymer is selected to control the drug delivery profile, such as to provide a more steady flux (e.g., a flatter drug flux curve). The impact of the acrylic polymer on the drug delivery profile may depend, for example, on the solubility of the drug in the acrylic polymer, with higher solubility correlated with a lower drug flux.

The term “acrylic polymer” is used here as in the art interchangeably with “polyacrylate,” “polyacrylic polymer,” and “acrylic adhesive.” The acrylic polymer can be any of the homopolymers, copolymers, terpolymers, and the like of various acrylic acids or esters. In some embodiments, the acrylic-based polymers are adhesive polymers. In other embodiments, the acrylic-based polymers function as an adhesive by the addition of tackifiers, plasticizers, crosslinking agents or other additives. The acrylic polymer can be a copolymer, terpolymer or multipolymer. For example, the acrylic polymer can be any of the homopolymers, copolymers, terpolymers, and the like of various acrylic acids.

Suitable acrylic polymers include polymers of one or more monomers of acrylic acids and other copolymerizable monomers, copolymers of alkyl acrylates and/or methacrylates and/or copolymerizable secondary monomers or monomers with functional groups. Combinations of acrylic polymers based on their functional groups are also contemplated. Acrylic polymers having functional groups include copolymers and terpolymers which contain, in addition to nonfunctional monomer units, further monomer units having free functional groups. The monomers can be monofunctional or polyfunctional. By varying the amount of each type of monomer added, the cohesive properties of the resulting acrylic polymer can be changed as is known in the art. In some embodiments, the acrylic polymer is composed of at least 50% by weight of an acrylate or alkyl acrylate monomer, from 0 to 20% of a functional monomer copolymerizable with the acrylate, and from 0 to 40% of other monomers.

Acrylate monomers which can be used include acrylic acid and methacrylic acid and alkyl acrylic or methacrylic esters such as methyl acrylate, ethyl acrylate, propyl acrylate, amyl acrylate, butyl acrylate, butyl methacrylate, hexyl acrylate, methyl methacrylate, hexyl methacrylate, heptyl acrylate, octyl acrylate, nonyl acrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, glycidyl acrylate, and corresponding methacrylic esters.

Non-functional acrylic-based polymers can include any acrylic based polymer having no or substantially no free functional groups.

Functional monomers, copolymerizable with the above alkyl acrylates or methacrylates, which can be used include acrylic acid, methacrylic acid, maleic acid, maleic anhydride, hydroxyethyl acrylate, hydroxypropyl acrylate, acrylamide, dimethylacrylamide, acrylonitrile, dimethylaminoethyl acrylate, dimethylaminoethyl methacrylate, tert-butylaminoethyl acrylate, tert-butylaminoethyl methacrylate, methoxyethyl acrylate and methoxyethyl methacrylate.

As used herein, “functional monomers or groups,” are monomer units typically in acrylic-based polymers which have reactive chemical groups which modify the acrylic-based polymers directly or which provide sites for further reactions. Examples of functional groups include carboxyl, epoxy, hydroxyl, sulfoxyl, and amino groups. Acrylic-based polymers having functional groups contain, in addition to the nonfunctional monomer units described above, further monomer units having free functional groups. The monomers can be monofunctional or polyfunctional. These functional groups include carboxyl groups, hydroxy groups, amino groups, amido groups, epoxy groups, etc. Typical carboxyl functional monomers include acrylic acid, methacrylic acid, itaconic acid, maleic acid, and crotonic acid. Typical hydroxy functional monomers include 2-hydroxyethyl methacrylate, 2-hydroxyethyl acrylate, hydroxymethyl acrylate, hydroxymethyl methacrylate, hydroxyethyl acrylate, hydroxyethyl methacrylate, hydroxypropyl acrylate, hydroxypropyl methacrylate, hydroxybutyl acrylate, hydroxybutyl methacrylate, hydroxyamyl acrylate, hydroxyamyl methacrylate, hydroxyhexyl acrylate, hydroxyhexyl methacrylate. As noted above, in some embodiments, the acrylic polymer does not include such functional groups.

Suitable acrylic polymers also include pressure-sensitive adhesives which are commercially available, such as the acrylic-based adhesives sold under the trademarks DURO-TAK® (such as DURO-TAK® 87-900A, 87-2516, 87-2287, -4098, -2852, -2196, -2296, -2194, -2516, -2070, -2353, -2154, -2510, -9085, -9088 and 73-9301) and GELVA® Multipolymer Solution (such as GELVA® 2480, 788, 737, 263, 1430, 1753, 1151, 2450, 2495, 3067, 3071, 3087 and 3235) both by Henkel Corporation, Bridgewater, N.J. Other suitable acrylic adhesives include those sold under the trademark EUDRAGIT® by Evonik Industries AG Pharma Polymers, Darmstadt, Germany.

In some embodiments, the compositions comprise a hydroxyl functional acrylic polymer (i.e., an acrylic polymer that includes hydroxy functional monomers), an amide functional acrylic polymer (i.e., an acrylic polymer that includes amide functional monomers), and/or a non-functional acrylic polymer. In some embodiments, the composition does not include a carboyyl-functional acrylic polymer (i.e., an acrylic polymer that includes carboxyl functional monomers).

For example, acrylic polymers that include hydroxy functional monomers generally exhibit good solubility for NETA, which impacts the drug delivery profile as illustrated in the examples, and permits sustained delivery of NETA over an extended period of time, such as a period of at least 3 days, at least 4 days, or at least 7 days, or longer. Hydroxy functional adhesives with a reactive functional OH group in the polymeric chain include GELVA® 737, 788, and 1151, and DURO-TAK® 87-2287, -4287-2510, -2516, 387-2510 and 387-2287.

Further details and examples of acrylic adhesives which are suitable in the practice of the invention are described in Satas, “Acrylic Adhesives,” Handbook of Pressure-Sensitive Adhesive Technology, 2nd ed., pp. 396-456 (D. Satas, ed.), Van Nostrand Reinhold, N.Y. (1989); “Acrylic and Methacrylic Ester Polymers,” Polymer Science and Engineering, Vol. 1, 2nd ed., pp 234-268, John Wiley & Sons, (1984); U.S. Pat. No. 4,390,520; and U.S. Pat. No. 4,994,267, all of which are expressly incorporated by reference in their entireties.

In embodiments comprising an acrylic polymer, one or more acrylic polymers can be used, and the acrylic polymer(s) may comprise from about 1% to about 15% by weight of the polymer matrix, including from about 5% to about 15%, including about 5%, about 10%, or about 15% by weight, based on the dry weight of the polymer matrix, e.g., not including the weight of processing solvents that are removed during the manufacturing process.

Penetration Enhancers

A “penetration enhancer” is an agent that accelerates the delivery of the drug through the skin. These agents also have been referred to as permeation enhancers, accelerants, adjuvants, and sorption promoters, and are collectively referred to herein as “enhancers.” This class of agents includes those with diverse mechanisms of action, including those which have the function of improving percutaneous absorption, for example, by changing the ability of the stratum corneum to retain moisture, softening the skin, improving the skin's permeability, acting as penetration assistants or hair-follicle openers or changing the state of the skin including the boundary layer.

Illustrative penetration enhancers include but are not limited to polyhydric alcohols such as dipropylene glycol, propylene glycol, and polyethylene glycol; oils such as olive oil, squalene, and lanolin; fatty ethers such as cetyl ether and oleyl ether; fatty acid esters such as isopropyl myristate; urea and urea derivatives such as allantoin which affect the ability of keratin to retain moisture; polar solvents such as dimethyidecylphosphoxide, methyloctylsulfoxide, dimethyllaurylamide, dodecylpyrrolidone, isosorbitol, dimethylacetonide, dimethylsulfoxide, decylmethylsulfoxide, and dimethylformamide which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the skin and drugs administered. Other agents include oleic and linoleic acids, ascorbic acid, panthenol, butylated hydroxytoluene, tocopherol, tocopheryl acetate, tocopheryl linoleate, propyl oleate, and isopropyl palmitate.

In some embodiments, the penetration enhancer component includes one or more of oleyl alcohol, dipropylene glycol, isopropyl myristate, and glyceryl monooleate, including combinations of any two or more of these, such as, for example, combinations of oleyl alcohol and dipropylene glycol; oleyl alcohol and glyceryl monooleate; and dipropylene glycol, isopropyl myristate, and glyceryl monooleate.

In some embodiments, the penetration enhancer component comprises from about 1% to about 15% by weight of the polymer matrix, including about 1% to about 5%, including about 1%, about 2%, about 3%, about 4%, about 5%, about 6%, about 7%, about 8%, about 9%, or about 10% by weight, based on the dry weight of the polymer matrix, e.g., not including the weight of processing solvents that are removed during the manufacturing process.

The amount of a given penetration enhancer can be selected by a person skilled in the art, based on the properties of the enhancer and the target drug delivery profile (e.g., rate and duration of drug delivery). For example, when oleyl alcohol and dipropylene glycol are used, the oleyl alcohol may comprise about 2% by weight of the polymer matrix and the dipropylene glycol may comprise about 8% by weight of the polymer matrix, based on the dry weight of the polymer matrix. In another example, when glyceryl monooleate, isopropyl myristate and dipropylene glycol are used, the glyceryl monooleate may comprise about 2% by weight of the polymer matrix, the isopropyl myristate may comprise about 2% by weight of the polymer matrix, and the dipropylene glycol may comprise about 8% by weight of the polymer matrix, based on the dry weight of the polymer matrix. In another example, when oleyl alcohol and glyceryl monooleate are used, the oleyl alcohol may comprise about 2% by weight of the polymer matrix and the glyceryl monooleate may comprise about 2% by weight of the polymer matrix, based on the dry weight of the polymer matrix. In another example, oleyl alcohol is used and comprises about 2% by weight of the polymer matrix, based on the dry weight of the polymer matrix. These examples are illustrative only and not limiting of the compositions described herein.

Crystallization Inhibitor

The polymer matrix may include one or more crystallization inhibitors that inhibit the crystallization of norethrindone acetate formulated in the matrix. Suitable crystallization inhibitors include one or more of povidone, polyvinylpyrrolidone (PVP), copovidone, polyvinylpyrrolidone-vinyl acetate copolymer (PVP/VA), crospovidone, and polyvinylpolypyrrolidone (PVPP). Specific examples include the following BASF (Germany) products: KOLLIDON® K-30 (PVP), KOLLIDON® CL (cross-linked PVP) and KOLLIDON® VA-64 (PVP/VA copolymer).

The amount of crystallization inhibitor(s) can be selected by a person skilled in the art, based on the properties of the inhibitor and desired effect, such as an amount effective to suppress the crystallization of NETA. In some embodiments, a crystallization inhibitor is used in an amount up from about 5% to about 25% by weight, based on the dry weight of the polymer matrix, including from about 10% to about 20% by weight, such as about 5%, as about 10%, about 15%, about 20% by weight, or about 25% by weight.

Other Components

The polymer matrix may include one or more other components, such as one or more other pharmaceutically acceptable excipients, such as an antioxidant, plasticizer, filler, and the like. In some embodiments, the polymer matrix comprises from about 0% to about 20% by dry weight of one or more such excipients.

In some embodiments, the polymer matrix may comprise, on a dry weight/weight basis, about 1% to about 5% NETA; about 60% to about 75% adhesive polymer (including 0 to about 10% acrylic polymer), about 1% to about 10% penetration enhancer, and about 10% to about 25% crystallization inhibitor.

Face Adhesive

As noted above, in some embodiments, the transdermal drug delivery system comprises a skin-contacting face adhesive layer separate from the polymer matrix layer. In accordance with such embodiments, the face adhesive may comprise any one or more of the polymers described above.

In any embodiments, the face adhesive may comprise a silicone polymer, such as one or more of those described above. In some embodiments, the face adhesive is a silicone face adhesive comprising a silicone adhesive, such as a silicone pressure-sensitive adhesive and, optionally, one or more penetration enhancers, such as one or more of those discussed above. In specific embodiments, the face adhesive comprises, on a dry weight/weight basis, from about 90 to about 100% of a silicone adhesive polymer and from about 0 to about 10% of one or more penetration enhancers.

Additionally or alternatively, in any embodiments, the face adhesive may comprise an acrylic polymer, such as a non-functional acrylic polymer, including one or more of Duro-Tak® 87-9900, -900A, -9301, -4098 or GELVA® GMS 3235, 3083, or 3252.

Additionally or alternatively, in any embodiments, the face adhesive may comprise a PIB polymer, such as any of the PIB polymers (or mixtures thereof) described above.

In some embodiments, the polymer component(s) of the face adhesive are selected to achieve one or more of the following characteristics: sustained drug delivery; good skin adhesion for the intended period of application (e.g., at least 1 day, at least 3 days, at least 7 days, or longer); minimal resistance to drug diffusion; minimal solubility for the drug without exhibiting “dumping” upon initial contact with skin that leads to a “burst effect;” physical and chemical compatibility with the drug. As illustrated in the examples, embodiments having a face adhesive exhibit good drug delivery, including sustained drug delivery over at least 3 days and at least 7 days.

As noted above, in some embodiments, the transdermal drug delivery system is a monolithic transdermal drug delivery system that does not include, for example, a face adhesive.

Transdermal Delivery Systems

The transdermal delivery systems may be of any shape or size suitable for transdermal application.

The drug-in-adhesive polymer matrices described herein may be prepared by methods known in the art. For example, the polymer matrix material can be applied to a backing layer and release liner by methods known in the art, and formed into sizes and shapes suitable for use. For example, after the polymer matrix is formed, it may be brought into contact with a support layer, such as a releaser liner layer or backing layer, in any manner known to those of skill in the art. Such techniques include calender coating, hot melt coating, solution coating, etc.

For example, a polymer matrix can be prepared by blending the components of the polymer matrix in the presence of a solvent, such as a volatile organic solvent, applying the wet blend of matrix material to a support layer such as a backing layer or release liner, removing any remaining solvents, and laminating to a release line or backing layer. The NETA can be added at any stage. In one embodiment, all polymer matrix components, including NETA, are blended together. In another embodiment, the polymer matrix components other than NETA are blended together, and then the NETA is dissolved or dispersed therein. The order of steps, amount of ingredients, and the amount and time of agitation or mixing can be determined and optimized by the skilled practitioner.

When a face adhesive is used, the face adhesive layer can be prepared separately by a similar method, and laminated to a polymer matrix layer provided on a backing layer (such as a polymer matrix layer formed on a backing layer.) A release liner may be provided on the skin-contacting side of the face adhesive layer, such as by forming the face adhesive layer on a release liner, or by applying a release liner to a preformed face adhesive layer

Individual units can be cut from a laminate produced as described above and packaged in a pouchstock, as discussed above.

Therapeutic Methods

In some embodiments, there is provided a method of effecting transdermal drug delivery of NETA by applying a system as described herein to the skin or mucosa of a subject in need thereof. In some embodiments, the system is applied over a period of at least about 1 day, at least about 2 days, at least about 3 days, at least about 4 days, at least about 5 days, at least about 6 days, or at least about 7 days, such as for 1, 2, 3, 4, 5, 6 or 7 days. In some embodiments, the method is effective to achieve therapeutic levels of NETA in the subject during the application period, such as levels effective to achieve contraception. In specific embodiments, the transdermal system is formulated to deliver at least about 0.14 mg/day or at least about 0.25 mg/day NETA during the application period.

In some embodiments, the systems described herein are designed for use by female patients, such as for contraception.

The following specific examples are included as illustrative of the transdermal drug delivery systems and polymer matrices described herein. These examples are in no way intended to limit the scope of the invention. Other aspects of the invention will be apparent to those skilled in the art to which the invention pertains.

### Examples 1-3

Monolithic systems comprising a silicone polymer matrix with or without a hydoxy-functional acrylic polymer (GELVA® 788) having the following polymer matrix compositions are prepared as follows:

| Components (% w/w) | Ex. 1 | Ex. 2 | Ex. 3 |
|---|---|---|---|
| NETA | 1.5 | 1.5 | 1.5 |
| Silicone Polymer | 73.50 | 68.5 | 63.5 |
| PVP K-30 | 15.0 | 15.0 | 15.0 |
| Oleyl Alcohol | 2.0 | 2.0 | 2.0 |
| Dipropylene Glycol | 8.0 | 8.0 | 8.0 |
| Acrylic Polymer | — | 5.0 | 10.0 |

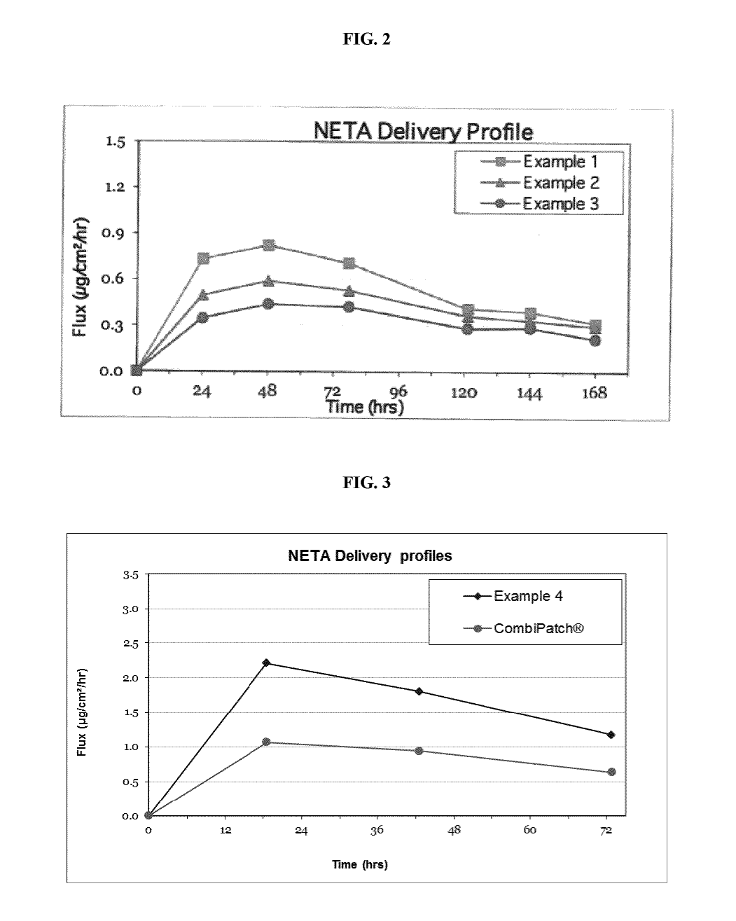

FIG. 2 reports the in vitro flux of NETA from the systems over 7 days, as assessed using human cadaver skin and a Franz cell set up. Results show that the presence and amount of the hydroxyl-functional acrylic polymer impacts the drug delivery profile, providing more controlled, sustained release (as reflected in the flatter profile of the flux curve). Thus, the type and amount of acrylic polymer can be selected and controlled to select and control drug delivery profile.

The formulation of Example 1 exhibited a NETA flux higher than that of CombiPatch®, the formulation of Example 2 exhibited a NETA flux comparable to that of CombiPatch®, and the formulation of Example 3 exhibited a NETA flux lower than that of CombiPatch®.

### Example 4

A monolithic system comprising a silicone polymer matrix having the following polymer matrix composition is prepared as follows:

| Components (% w/w) | Ex. 4 |
|---|---|
| NETA | 1.5 |
| Silicone Polymer | 71.50 |
| PVP K-30 | 15.0 |
| Glyceryl monooleate | 2.0 |
| Isopropyl Myristate | 2.0 |
| Dipropylene Glycol | 8.0 |

FIG. 3 reports the in vitro flux of NETA from the system over 7 days as compared to the flux of NETA from CombiPatch®, as assessed using human cadaver skin and a Franz cell set up. Results show that the system achieved a drug flux about two times that of CombiPatch®.

### Examples 5-6

PIB polymer matrix compositions are prepared as follows, and formed into systems without or with a face adhesive layer:

| Components (% w/w) | Ex. 5 | Ex. 6 |
|---|---|---|
| NETA | 1.5 | 1.5 |
| PIB Polymer | 74.50 | 74.5 |
| PVP K-30 | 20.0 | 20.0 |
| Oleyl alcohol | 2.0 | 2.0 |
| Glyceryl monooleate | 2.0 | 2.0 |
| Face Adhesive | — | PIB |

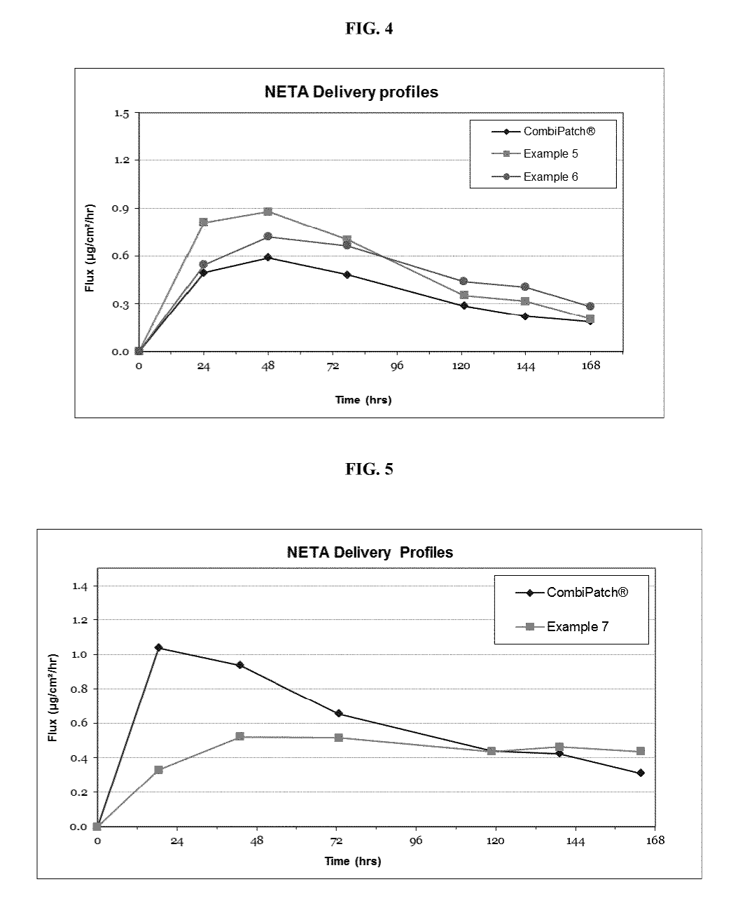

FIG. 4 reports the in vitro flux of NETA from the system over 7 days as compared to the flux of NETA from CombiPatch®, as assessed using human cadaver skin and a Franz cell set up. Results show that the PIB system prepared without a face adhesive layer delivered NETA about 30% faster than of CombiPatch®, while the PIB system prepared with a face adhesive layer achieved a more constant and sustained drug flux (as reflected in the flatter drug flux curve) at a higher level than CombiPatch®.

### Example 7

A system comprising a PIB polymer matrix having the following polymer matrix composition and a silicone face adhesive is prepared as follows:

| Components (% w/w) | Ex. 7 |
|---|---|
| NETA | 5.0 |
| PIB Polymer | 73.0 |
| PVP VA-64 | 20.0 |
| Oleyl Alcohol | 2.0 |

FIG. 5 reports the in vitro flux of NETA from the system over 7 days as compared to the flux of NETA from CombiPatch®, as assessed using human cadaver skin and a Franz cell set up. Results show that the system of Example 7 achieved a sustained drug flux over seven days, lower than that of CombiPatch®, but still at levels believed to be correlated with therapeutic effect.

### Example 8

A system comprising a PIB polymer matrix with a hydroxyl-functional acrylic polymer (GELVA® 788) having the following polymer matrix composition and a silicone face adhesive is prepared as follows:

| Components (% w/w) | Ex. 7 |
|---|---|
| NETA | 2 |
| PIB Polymer | 69.0 |
| Acrylic Polymer | 5% |
| K-CL-M* | 20.0 |
| Oleyl Alcohol | 2.0 |
| Glyceryl monooleate | 2.0 |

*K-CL-M is micronized KOLLIDON ® CL.

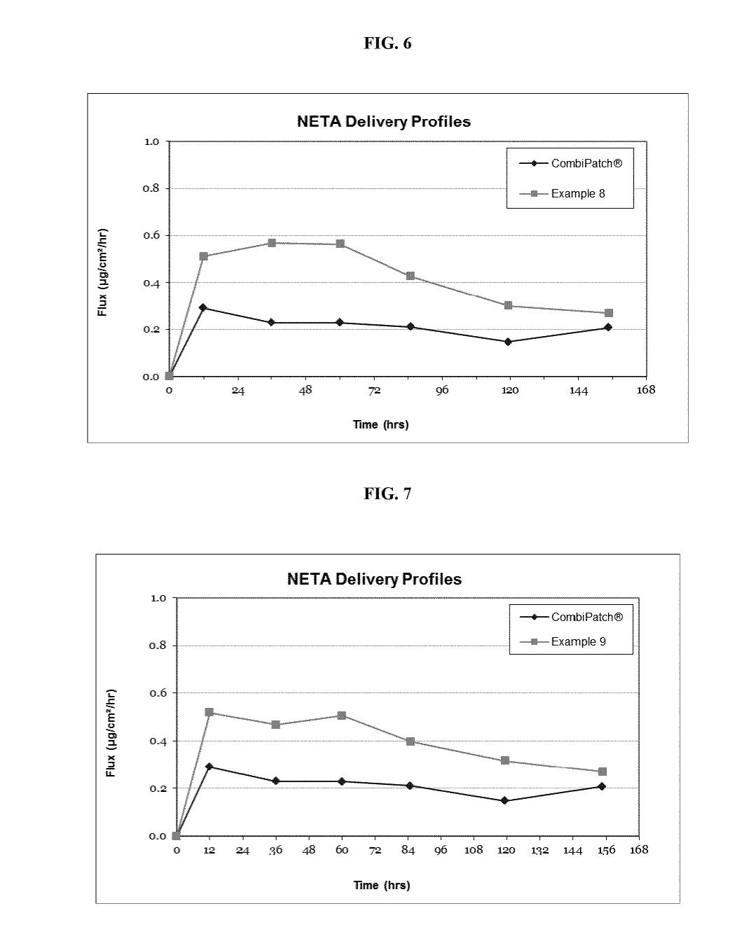

FIG. 6 reports the in vitro flux of NETA from the system over 7 days as compared to the flux of NETA from CombiPatch®, as assessed using human cadaver skin and a Franz cell set up. Results show that the system of Example 8 achieved a sustained drug flux over seven days, that was about twice that of CombiPatch®.

### Example 9

A system comprising a PIB polymer matrix with an amide-containing acrylic polymer (DURO-TAK® 87-900A) having the following polymer matrix composition and a silicone face adhesive is prepared as follows:

| Components (% w/w) | Ex. 7 |
|---|---|
| NETA | 2 |
| PIB Polymer | 69.0 |
| Acrylic Polymer | 5% |
| K-CL-M* | 20.0 |
| Oleyl Alcohol | 2.0 |
| Glyceryl monooleate | 2.0 |

*K-CL-M is micronized KOLLIDON ® CL.

FIG. 7 reports the in vitro flux of NETA from the system over 7 days as compared to the flux of NETA from CombiPatch®, as assessed using human cadaver skin and a Franz cell set up. Results show that the system of Example 9 achieved a sustained drug flux over seven days, that was about 1.5 times that of CombiPatch®.

What is claimed is:

1. A transdermal drug delivery system for the transdermal delivery of norethindrone acetate in the form of a flexible finite system for topical application, comprising a drug-in-adhesive polymer matrix comprising norethindrone acetate (NETA) as the only systemically active drug, an adhesive polymer selected from silicone adhesives and polyisobutylene (PIB) adhesives, a penetration enhancer, and a crystallization inhibitor.

2. The transdermal drug delivery system of claim 1, wherein the penetration enhancer is selected from the group consisting of oleyl alcohol, dipropylene glycol, isopropyl myristate, glyceryl monooleate, and combinations thereof.

3. The transdermal drug delivery system of claim 2, wherein the penetration enhancer comprises oleyl alcohol.

4. The transdermal drug delivery system of claim 2, wherein the penetration enhancer comprises oleyl alcohol and dipropylene glycol.

5. The transdermal drug delivery system of claim 2, wherein the penetration enhancer comprises oleyl alcohol and glyceryl monooleate.

6. The transdermal drug delivery system of claim 2, wherein the penetration enhancer comprises dipropylene glycol, ispropyl myristate and glyceryl monooleate.

7. The transdermal drug delivery system of claim 1, wherein the crystallization inhibitor is selected from the group consisting of povidone, polyvinylpyrrolidone (PVP), copovidone, polyvinylpyrrolidone-vinyl acetate copolymer (PVP/VA), crospovidone, and polyvinylpolypyrrolidone (PVPP).

8. The transdermal drug delivery system of claim 1, wherein the polymer comprises a silicone pressure-sensitive adhesive polymer.

9. The transdermal drug delivery system of claim 1, wherein the polymer comprises a PIB pressure-sensitive adhesive polymer.

10. The transdermal drug delivery system of claim 1, wherein the polymer matrix further comprises an acrylic polymer.

11. The transdermal drug delivery system of claim 10, wherein the acrylic polymer is selected from the group consisting of non-functional acrylic polymers, hydroxyl-functional acrylic polymers and amide-functional acrylic polymers.

12. The transdermal drug delivery system of claim 1, wherein the polymer matrix comprises, on a dry weight/weight basis, about 1 to about 2.5 NETA; about 60% to about 75% adhesive polymer, about 1% to about 10% penetration enhancer, and about 10% to about 25% crystallization inhibitor.

13. The transdermal drug delivery system of claim 1, wherein the system is a monolithic system.

14. The transdermal drug delivery system of claim 1, wherein the system further comprises a face adhesive layer.

15. The transdermal drug delivery system of claim 14, wherein the face adhesive layer is a polyisobutylene face adhesive layer.

16. The transdermal drug delivery system of claim 1, wherein the system further comprises a backing layer.

17. The transdermal drug delivery system of claim 1, wherein the system further comprises a release liner.

18. The transdermal drug delivery system of claim 1, wherein the polymer matrix comprises an amount of NETA effective to deliver a pharmaceutically effective amount of NETA for contraception over a period of time of at least 3 days, at least 4 days, or at least 7 days.

19. The transdermal drug delivery system of claim 1, wherein the polymer matrix comprises an amount of NETA effective to deliver at least about 0.14 mg/day NETA over a period of time of at least 3 days, at least 4 days, or at least 7 days.

20. The transdermal drug delivery system of claim 1, wherein the polymer matrix comprises an amount of NETA effective to deliver at least about 0.25 mg/day NETA over a period of time of at least 3 days, at least 4 days, or at least 7 days.

21. A method of preparing a transdermal composition for the transdermal delivery of norethindrone acetate in the form of a flexible finite system for topical application, comprising preparing a drug-in-adhesive polymer matrix comprising norethindrone acetate (NETA) as the only systemically active drug, an adhesive polymer selected from silicone adhesives and polyisobutylene (PIB) adhesives, a penetration enhancer, and a crystallization inhibitor.

22. A method of transdermally delivering norethindrone acetate, comprising applying a transdermal drug delivery system of claim 1 to the skin or mucosa of a subject in need thereof.

23. A method of providing contraception to a human female subject in need thereof, comprising applying a transdermal drug delivery system of claim 1 to the skin or mucosa of the subject in need thereof.

24. The method of claim 23, wherein the polymer matrix of the transdermal drug delivery system comprises an amount of NETA effective to deliver a pharmaceutically effective amount of NETA for contraception over a period of time of at least 3 days, at least 4 days, or at least 7 days, and the system is applied for a duration of at least 3 days, at least 4 days, or at least 7 days, respectively.
